(19) United States
(12) Patent Application Publication
(10) Pub. No.: US 2016/0015942 A1
(43) Pub. Date: Jan. 21, 2016

(54) CATHETER ASSEMBLY

(71) Applicants: Terumo Kabushiki Kaisha, Tokyo (JP); Mitsubishi Pencil Company, Limited, Tokyo (JP)

(72) Inventors: Yuya TERASAWA, Nakakoma-gun (JP); Yasunobu ZUSHI, Nakakoma-gun (JP)

(73) Assignees: Terumo Kabushiki Kaisha, Tokyo (JP); Mitsubishi Pencil Company, Limited, Tokyo (JP)

(21) Appl. No.: 14/871,440

(22) Filed: Sep. 30, 2015

Related U.S. Application Data
(63) Continuation of application No. PCT/JP2013/059812, filed on Apr. 1, 2013.

(51) Int. Cl. A61M 25/06

(52) U.S. Cl. CPC A61M 25/0618; A61M 25/0606

(57) ABSTRACT

A catheter assembly includes an inner needle having a sharp tip at a distal end thereof; a catheter in which the inner needle is disposed; a catheter hub disposed on a proximal end side of the catheter and through which the inner needle is insertable; an inner needle hub disposed on a proximal end side of the inner needle; and a protector mounted detachably on the inner needle hub, the protector being configured to cover at least the tip of the inner needle. The protector includes a movement prevention unit, the movement prevention unit being configured to prevent movement of the catheter toward the tip with respect to the inner needle in a state in which a distal end side of the inner needle protrudes a predetermined length from the catheter.

### CROSS-REFERENCE TO RELATED PATENT APPLICATION

This application is a continuation application of and claims the benefit of priority from International Patent Application No. PCT/JP2013/059812 filed on Apr. 1, 2013, the contents of which are incorporated herein by reference.

### TECHNICAL FIELD

The present disclosure relates to a catheter assembly that punctures and remains indwelling in a blood vessel when, for example, performing a blood transfusion on a patient.

### BACKGROUND

Conventionally, when a blood transfusion is carried out on a patient, for example, a catheter assembly is used. The catheter assembly is typically equipped with an inner needle having a sharp tip at a distal end thereof, a catheter (outer needle) through which the inner needle is inserted, a catheter hub that is fixed to a proximal end of the catheter and through which the inner needle is inserted, and an inner needle hub that is fixed to a proximal end of the inner needle.

In the event that the catheter assembly is used to perform a blood transfusion on a patient, in a state in which the distal end side of the inner needle protrudes outwardly from the catheter, the inner needle, together with the catheter, punctures a blood vessel of a patient. After puncturing, the inner needle is withdrawn from the catheter while the catheter remains indwelling in the blood vessel. Thereafter, a connector, which is provided on a distal end of an infusion tube, is connected to the proximal end of the catheter hub, and an infusion solution is supplied into the patient's blood vessel through an infusion tube, the catheter hub, and the catheter.

As a catheter assembly of this type, a catheter assembly has been proposed that is equipped with a protective sheath that is detachably attached to the inner needle hub in a state in which the tip and the catheter are accommodated therein (see, for example,

### SUMMARY

In the catheter assembly, in order to facilitate insertion of the catheter into a blood vessel of the patient, it is necessary for a distal end side of the inner needle to protrude outwardly by a predetermined length from the catheter.

However, because the relative movement in the axial direction between the inner needle and the catheter is basically only limited by the frictional force between the inner needle and the catheter, there is a concern that the length by which the inner needle protrudes with respect to the catheter may be changed due to relative movement in the axial direction between the inner needle and the catheter during, for example, transportation, sterilization, or the like of the catheter assembly.

Certain embodiments of the present invention have been devised taking into consideration the aforementioned difficulties, and has an object of certain embodiments of providing a catheter assembly in which, in an initial condition prior to use thereof, the amount at which the inner needle protrudes from the catheter can be held constant.

A catheter assembly according to an embodiment of the present invention includes an inner needle having a sharp tip at a distal end thereof, a catheter through which the inner needle is inserted, a catheter hub disposed on a proximal end side of the catheter and through which the inner needle is inserted, an inner needle hub disposed on a proximal end side of the inner needle, and a protector mounted detachably on the inner needle hub. The protector is configured to cover at least the tip and is provided with a movement prevention unit. The movement prevention unit is configured to prevent movement of the catheter toward the tip with respect to the inner needle in a state in which a distal end side of the inner needle protrudes a predetermined length from the catheter.

According to an embodiment of the catheter assembly of the present invention, the movement prevention unit is provided on the protector for preventing movement of the catheter toward the tip with respect to the inner needle, in a condition in which the distal end side of the inner needle protrudes a predetermined length from the catheter. Thus, in an initial state prior to use thereof (when the protector is mounted on the inner needle hub), during transportation, sterilization, or the like of the catheter assembly, relative movement between the catheter and the inner needle in the axial direction can be suppressed. Consequently, in the initial state, the length at which the inner needle protrudes from the catheter can be held constant.

In the aforementioned catheter assembly, a tab may project from an outer surface of the catheter hub, and the movement prevention unit may include a tab abutment configured to abut against a surface of the tab that is oriented toward the tip.

According to such an arrangement, with a simple structure in which the tab abutment is made to abut against a surface of the tab of the catheter hub that is oriented toward the tip, movement of the catheter toward the tip with respect to the inner needle can be prevented.

In the above-described catheter assembly, the protector may include a hollow mounting portion that is detachably fitted externally over the inner needle hub, and a fitting projection configured to contact the inner needle hub may be formed in a projecting manner on an inner surface of the mounting portion.

According to such an arrangement, because the fitting projections that contact the inner needle hub are formed to project outwardly on the inner surface of the mounting portion that makes up part of the protector, the mounting portion can be fitted externally over the inner needle hub with a suitable fitting force. Consequently, in the initial state, because the protector can be prevented from being displaced along the axial direction of the inner needle with respect to the inner needle hub, the length at which the inner needle protrudes from the catheter can more reliably be held constant. Further, during use thereof, the protector can be detached from the inner needle hub with an appropriate force.

The fitting projection may be formed to become narrower in the projecting direction of the fitting projection.

According to this structure, because the fitting projections are formed to become narrower in the projecting direction thereof, the fitting force with respect to the inner needle hub can be suppressed appropriately.

In the aforementioned catheter assembly, the fitting projection may include an outer surface that is arcuately shaped in cross-section.

According to this structure, the fitting projections include outer surfaces that are arcuately shaped in cross-section, and thus, while the fitting force with respect to the inner needle hub is appropriately suppressed, the rigidity of the fitting projections can be suitably increased. Therefore, a configuration can be provided in which it is unlikely that the fitting projections will become damaged.

In the aforementioned catheter assembly, the fitting projection may extend along an axial direction of the inner needle.

In accordance with this structure, the fitting projections extend along the axial direction of the inner needle, and thus, at an initial stage in which the mounting portion is fitted externally over the inner needle hub, the frictional force between the fitting projections and the inner needle hub can be kept comparatively small. Consequently, it is easy for the mounting portion to be fitted externally over the inner needle hub and an appropriate fitting force can be produced in a state in which the mounting portion is fitted completely over the inner needle hub.

In the above-described catheter assembly, the fitting projection may include a plurality of fitting projections, and the fitting projections may face each other and sandwich the inner needle hub therebetween.

According to such a structure, because the plural fitting projections face each other mutually while sandwiching the inner needle hub therebetween, the inner needle hub can be gripped by the fitting projections. Thus, with a simple structure, an appropriate fitting force can be generated between the mounting portion and the inner needle hub.

In the above-described catheter assembly, the inner needle hub may include a first engaging portion, the first engaging portion may be configured to guide the mounting portion along an axial direction of the inner needle, and the mounting portion may include a second engaging portion formed on the inner surface thereof, the second engaging portion may be configured to engage with the first engaging portion.

According to such a structure, by moving the mounting portion and the inner needle hub along the axial direction of the inner needle in a state in which the first engaging portion and the second engaging portion are engaged, the mounting portion can be easily attached and detached with respect to the inner needle hub.

In the aforementioned catheter assembly, either one of the first engaging portion and the second engaging portion may be a protrusion that projects substantially perpendicularly to the projecting direction of the fitting projection, and the other one of the first engaging portion and the second engaging portion may be a protrusion or a recess.

According to the above configuration, since either one of the first engaging portions and the second engaging portions are protrusions that project substantially perpendicularly to the projecting direction of the fitting projections, whereas the other one of the first engaging portions and the second engaging portions are protrusions or recesses, the inner needle hub can be gripped by the fitting projections and the second engaging portions. Thus, with a simple structure, an appropriate fitting force can be generated between the mounting portion and the inner needle hub.

In the above-described catheter assembly, the inner needle hub may include a projection, the projection may project in a radially outward direction of the inner needle, and the mounting portion may include a movement restricting portion formed on the inner surface thereof. The movement restricting portion may be configured to engage with the projection and thereby restrict movement of the inner needle hub toward the proximal end side with respect to the protector in a state in which the mounting portion is fitted externally over the inner needle hub.

According to such a structure, in a state in which the mounting portion is fitted externally over the inner needle hub, the movement restricting portions are formed, which engage with the projections of the inner needle hub and thereby restrict movement of the inner needle hub toward the proximal end side with respect to the protector, and thus, in the initial state, any change in the length by which the inner needle protrudes from the catheter can be suitably prevented.

The aforementioned catheter assembly may further include a needle cover member that is housed in the inner needle hub in a condition capable of expanding toward the distal end side so as to cover the tip, and the catheter hub may be disposed detachably with respect to the needle cover member.

According to such a structure, after the catheter has been left indwelling in the blood vessel, when the inner needle is withdrawn or pulled out, because the tip can be covered by the needle cover member, the user inadvertently touching the inner needle can be prevented. Further, even with a catheter assembly in which such a needle cover member is provided, in the initial state, the length at which the inner needle protrudes from the catheter can be held constant.

In the aforementioned catheter assembly, the movement prevention unit may include a needle cover member abutment configured to abut against a surface of the needle cover member that is oriented toward the tip.

According to such an arrangement, with a simple structure in which the needle cover member abutments are made to abut against a surface of the needle cover member that is oriented toward the tip, movement of the catheter toward the tip with respect to the inner needle can be reliably prevented.

### BRIEF DESCRIPTION OF THE DRAWINGS

FIG. 1 is a perspective view showing a catheter assembly (a first catheter assembly) according to a first embodiment of the present invention.

FIG. 2 is a perspective view of a catheter indwelling device constituting part of the first catheter assembly.

FIG. 3 is a perspective view as observed from a proximal end side of a protector that constitutes part of the first catheter assembly.

FIG. 4 is a vertical cross-sectional partial view of the first catheter assembly.

FIG. 5 is a cross-sectional view taken along a line V-V of FIG. 4.

FIG. 6 is a plan view showing in outline form the first catheter assembly.

FIG. 7 is a side view showing in outline form the first catheter assembly.

FIG. 8 is a rear view showing in outline form the first catheter assembly.

FIG. 9 is a perspective view showing a catheter assembly (a second catheter assembly) according to a second embodiment of the present invention.

FIG. 10 is a perspective view of a catheter indwelling device constituting part of the second catheter assembly.

FIG. 11 is a perspective view as observed from a proximal end side of a protector that constitutes part of the second catheter assembly.

FIG. 12 is a vertical cross-sectional partial view of the second catheter assembly.

FIG. 13 is a cross sectional view taken along line XIII-XIII of FIG. 12.

FIG. 14 is a plan view showing in outline form the second catheter assembly.

FIG. 15 is a side view showing in outline form the second catheter assembly.

### DETAILED DESCRIPTION OF THE EMBODIMENTS

Below, preferred exemplary embodiments of a catheter assembly according to the present invention will be presented in detail with reference to the accompanying drawings.

### First Embodiment

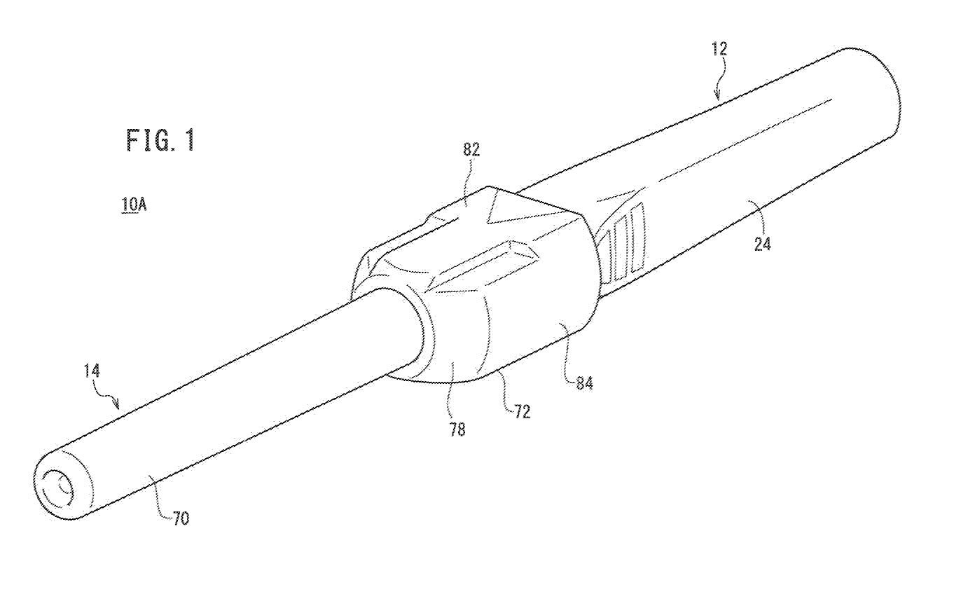

As shown in FIG. 1, a catheter assembly 10A is equipped with a catheter indwelling device 12 constituting a main body portion thereof, and a protector 14 that is detachably mounted on the catheter indwelling device 12.

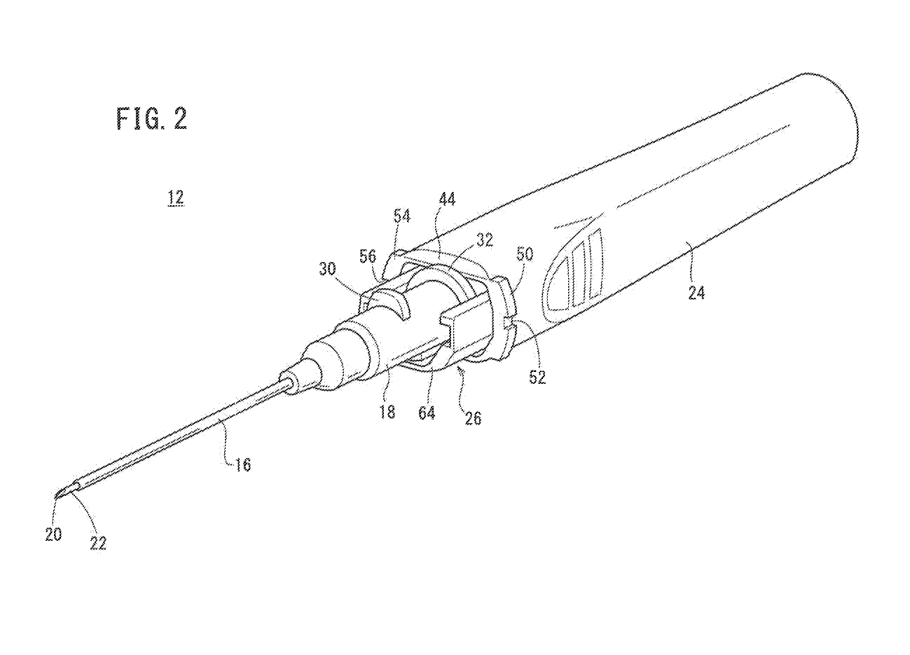

As shown in FIG. 2, the catheter indwelling device 12 is equipped with a tubular catheter 16 that functions as an outer needle, a catheter hub 18 that is connected to a proximal end side of the catheter 16, a tubular inner needle 22 having a sharp tip 20 on a distal end thereof and which is capable of being inserted through the interior of the catheter 16 and the catheter hub 18, an inner needle hub 24 connected to a proximal end side of the inner needle 22, and a needle cover member 26 that covers the inner needle 22 when the inner needle 22 is retracted. The catheter indwelling device 12 may used in the following manner, described in detail below.

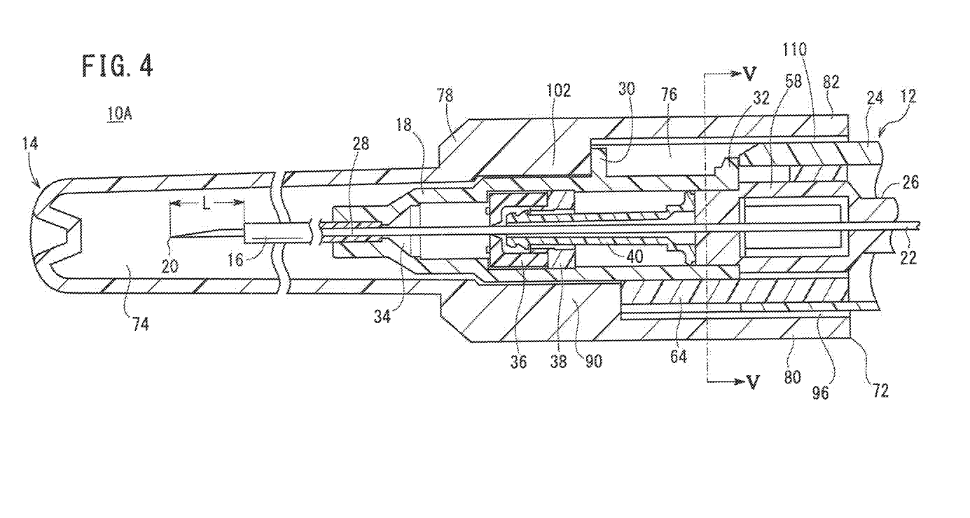

A user (e.g., a doctor or nurse) performs an operation by gripping the inner needle hub 24 of the catheter indwelling device 12, whereby the distal end portion thereof punctures and is inserted into a blood vessel of a patient. With the catheter indwelling device 12, in a condition prior to puncturing the patient (hereinafter a “punctured enabled state”), the inner needle 22 is inserted through the interior of the catheter 16 to form a double tube structure, in which the inner needle 22 protrudes a predetermined length L from the distal end of the catheter 16 (see FIG. 4). Further, in the puncture enabled state of the catheter indwelling device 12, the proximal end side of the catheter hub 18 is mounted on the distal end portion of the needle cover member 26 that projects from the distal end side of the inner needle hub 24.

In the catheter indwelling device 12, the catheter 16 and the inner needle 22, which make up the double tube structure, are inserted together into the blood vessel of the patient. After puncturing the patient, in a condition in which the position of the catheter 16 is maintained, by retracting the inner needle hub 24 in the direction of the proximal end, the needle cover member 26 is made to separate away from the catheter hub 18 and the inner needle 22, which is connected to the inner needle hub 24, is also pulled out integrally therewith and is separated from the catheter 16 and the catheter hub 18. As a result, among the elements of the catheter indwelling device 12, a state is brought about in which only the catheter 16 and the catheter hub 18 are left indwelling on the side of the patient.

When the inner needle 22 is withdrawn from the catheter 16, the inner needle 22 becomes accommodated inside the needle cover member 26 due to the needle cover member 26 extending to the distal end side with respect to the inner needle hub 24. Consequently, exposure of the inner needle 22 to the exterior is prevented. After the inner needle 22 has been withdrawn from the catheter 16, a non-illustrated connector of an infusion tube is connected to the proximal end side of the catheter hub 18, whereby an infusion agent (medicinal solution) may be supplied from the infusion tube to the patient.

Next, the structure of the catheter indwelling device 12 will be described. In the puncture enabled state, the catheter indwelling device 12 is constituted as a single assembly, in which the double tube structure of the catheter 16 and the inner needle 22, the catheter hub 18, the needle cover member 26, and the inner needle hub 24 are combined and are capable of being handled integrally.

As shown in FIGS. 2 and 4, the catheter 16, which is constituted as an outer needle in the catheter indwelling device 12, is a flexible tubular member formed with a predetermined length and having a narrow diameter. In the interior of the catheter 16, a lumen 28 is formed to extend and penetrate through the catheter 16 in the axial direction. The inner diameter (port diameter) of the lumen 28 is set to a size that enables the inner needle 22 to be inserted through the lumen 28.

The catheter 16 may be made from a resin material, in particular, a soft resin material. For example, a fluororesin, such as polytetrafluoroethylene (PTFE), ethylene-tetrafluoroethylene copolymer (ETFE), perfluoroalkoxy fluororesin (PFA), etc., an olefin resin, such as polyethylene, polypropylene, etc., or a mixture thereof, polyurethane, polyester, polyamide, polyether nylon resin, and a mixture of the olefin resin and ethylene-vinyl acetate copolymer may be used. In addition, the catheter 16 may be made from a transparent resin material such that all or a portion of the interior thereof is visible.

The catheter hub 18 is connected in a fixed manner to the proximal end of the catheter 16. The catheter hub 18 is formed with a tapering tubular (cylindrical) shape. A plate-shaped tab 30, which projects outwardly, is disposed integrally on the outer circumferential surface of the catheter hub 18. The tab 30 is formed with a size that can be pressed by the fingers of a user when removing the inner needle 22 from the catheter 16. A flange 32, which projects outwardly and extends in a circumferential direction, is provided on the proximal end of the catheter hub 18. In the puncture enabled state, the catheter hub 18 is detachably mounted on a distal end portion of the needle cover member 26.

When the catheter indwelling device 12 is used, the catheter hub 18 is exposed on the patient's skin in a state in which the catheter 16 has pierced into the blood vessel, and is pasted and held in place on the skin by tape or the like. The catheter hub 18 preferably is made from a material that is more rigid than the catheter 16. The constituent material of the catheter hub 18 is not limited to any particular material. However, a thermoplastic resin material, for example, polypropylene, polycarbonate, polyamide, polysulfone, polyarylate, methacrylate-butylene-styrene copolymer, etc., may be used.

In the interior of the catheter hub 18, there are arranged a crimp pin 34, which fixes together in a liquid-tight state the proximal end portion of the catheter 16 and the distal end portion of the catheter hub 18, a hemostasis valve 36, which prevents the flow of blood to the proximal end side of the catheter hub 18, a seal member 38, which is made from a material (e.g., a porous body) that allows for the flow of gas yet blocks the flow of liquid through the seal member 38, and a plug 40, which releases the hemostasis valve 36 when the connector of the infusion tube is connected.

The inner needle 22 is a rigid tubular member that is capable of puncturing the patient's skin. The inner needle 22 is sufficiently longer than the catheter 16 such that, in the puncture enabled state of the catheter indwelling device 12, the tip 20 of the inner needle 22 protrudes a predetermined length L from a distal end opening of the catheter 16. Further, in the puncture enabled state, an intermediate location in the longitudinal direction of the inner needle 22 is inserted through the interior of the catheter hub 18, and the proximal end side thereof is retained inside the inner needle hub 24. As the constituent material of the inner needle 22, a metal material, such as stainless steel, aluminum or aluminum alloy, titanium or titanium alloy, may be used.

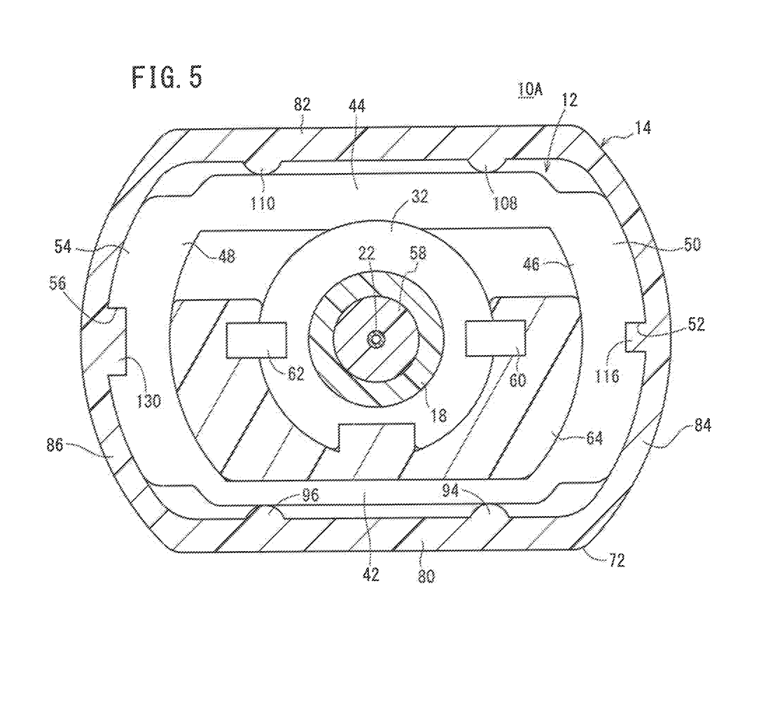

The inner needle hub 24 is a tubular member formed in a narrow elongate shape having a hollow portion of a predetermined volume. The inner needle hub 24 is formed with an appropriate size (thickness, length) to enable the inner needle hub 24 to be gripped and operated easily by the user when using the catheter indwelling device 12. As shown in FIG. 5, a distal end portion of the inner needle hub 24 includes a first flat plate portion 42 and a second flat plate portion 44, which face each other, and a first side wall 46 and a second side wall 48, which are curved arcuately outward and are connected to the first flat plate portion 42 and the second flat plate portion 44.

On the first side wall 46, a plate-shaped first projection 50 is formed integrally, which projects outwardly in the thickness direction (radially outward direction of the inner needle 22) of the first side wall 46. The first projection 50 is bent in an arcuate shape and, in a substantially center portion in the direction of extension thereof, a first guide groove (first engaging portion) 52 is formed along the axial direction of the inner needle 22. On the second side wall 48, a plate-shaped second projection 54 is formed integrally, which projects outwardly in the thickness direction (radially outward direction of the inner needle 22) of the second side wall 48. The second projection 54 is bent in an arcuate shape and, in a substantially center portion in the direction of extension thereof, a second guide groove (first engaging portion) 56 is formed along the axial direction of the inner needle 22. The first guide groove 52 and the second guide groove 56 serve to guide a mounting portion 72 along the axial direction of the inner needle 22, and the groove width of the second guide groove 56 is set to be greater than the groove width of the first guide groove 52.

The materials constituting the inner needle hub 24 are not particularly limited and may be, for example, the same materials as given in the description of the catheter hub 18. The same holds true for the needle cover member 26 and the protector 14. In this case, all of these members may be constituted from the same material, or may be constituted from different materials for each of the members.

As shown in FIGS. 4 and 5, in the puncture enabled state of the catheter indwelling device 12, the needle cover member 26 is accommodated and fixed in the inner needle hub 24 in a state (contracted state) enabling extension thereof toward the distal end side. On a distal end portion of the needle cover member 26, there are disposed a tip accommodating section 58, which includes a space formed therein that accommodates the tip 20 when the inner needle 22 has been retracted from the catheter 16, a pair of arms 60, 62, which are disposed on the tip accommodating section 58 and releasably engage with a flange 32 of the catheter hub 18, and an arm accommodating section 64 that accommodates the pair of arms 60, 62.

In a condition in which the pair of arms 60, 62 are subjected to no external force and thus are in a free state, the pair of arms 60, 62 expand outwardly in the direction of the distal end. More specifically, in the puncture enabled state, the pair of arms 60, 62 engage with the flange 32 of the catheter hub 18 in a state in which the outwardly expanded state of the arms 60, 62 is restrained by the arm accommodating section 64.

On the other hand, when the inner needle 22 is retracted from the catheter 16, while the catheter hub 18 is pressed and the inner needle hub 24 is retracted in the proximal direction, the pair of arms 60, 62 in engagement with the catheter hub 18 slide toward the distal end side beyond the arm accommodating section 64 and expand outwardly. Therefore, the state of engagement between the pair of arms 60, 62 and the flange 32 is released. Thus, the catheter hub 18 becomes disengaged from the needle cover member 26.

Next, the protector 14 that makes up part of the catheter assembly 10A will be described. As shown in FIG. 1, the protector 14 is an integrally molded article and, in an initial state (i.e., a state prior to use of the catheter assembly 10A, in which the protector 14 is mounted on the catheter indwelling device 12 in the puncture enabled state), the protector 14 serves to protect the distal end side of the catheter indwelling device 12. The protector 14 includes a protective tube 70 having a closed distal end, and a hollow mounting portion 72 disposed on the proximal end of the protective tube 70 and which makes up a proximal end side of the protector 14.

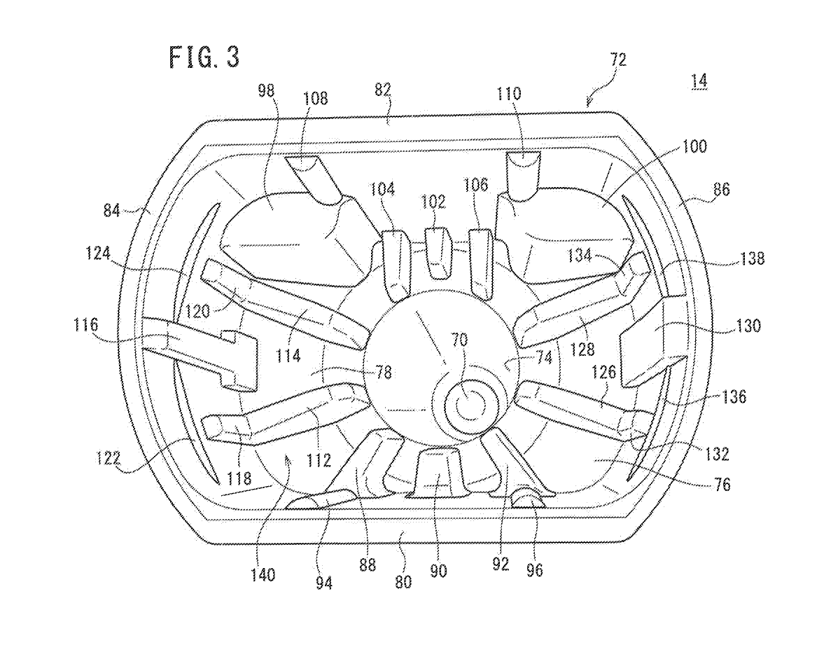

As shown in FIGS. 3 and 4, a lumen 74, in which the double tube structure of the inner needle 22 and the catheter 16 is accommodated, is formed in the protective tube 70. Another lumen 76, in which the catheter hub 18 is accommodated and into which the distal end portion of the inner needle hub 24 is inserted, is formed in the mounting portion 72.

The mounting portion 72 is mounted on the distal end side of the inner needle hub 24 of the catheter indwelling device 12. The mounting portion 72 includes a wall portion 78 that extends radially outward from the proximal end of the protective tube 70, a first flat plate portion 80 and a second flat plate portion 82 that face each other and extend from the wall portion 78 toward a side opposite to the protective tube 70, and a first side wall portion 84 and a second side wall portion 86, which are curved arcuately outward and are connected to the first flat plate portion 80 and the second flat plate portion 82.

On an inner surface of the first flat plate portion 80, there are formed, in an outwardly projecting manner, a plurality of (three in the present embodiment) needle cover member abutments 88, 90, 92 (also referred to as first needle cover member abutments 88, 90, 92) that abut against the needle cover member 26 of the catheter indwelling device 12, and a pair of fitting projections 94, 96 (also referred to as first fitting projections 94, 96) that contact the first flat plate portion 42 of the inner needle hub 24.

The first needle cover member abutments 88, 90, 92 are disposed at equal intervals in the widthwise direction (left and right direction in FIG. 3) of the first flat plate portion 80, and extend a predetermined length from the inner surface of the wall portion 78 toward the proximal end side. Each of the first needle cover member abutments 88, 90, 92 projects to the opening edge of the proximal end side of the protective tube 70. The first needle cover member abutment 90 that is positioned in the center is formed to be thicker than the first needle cover member abutments 88, 92 that are positioned on both sides thereof

Each of the first fitting projections 94, 96 extends along the direction of extension of the protective tube 70 (the axial direction of the inner needle 22). More specifically, the first fitting projection 94 extends from the proximal end of the first needle cover member abutment 88 to the proximal end of the first flat plate portion 80, and the first fitting projection 96 extends from the proximal end of the first needle cover member abutment 92 to the proximal end of the first flat plate portion 80. The amount at which each of the first fitting projections 94, 96 projects is set sufficiently shorter than the amount at which each of the first needle cover member abutments 88, 90, 92 project. Each of the first fitting projections 94, 96 is formed to become narrower in the projecting direction thereof More specifically, each of the first fitting projections 94, 96 is formed with a substantially semicircular shape in cross-section, and includes an outer surface that is arcuately shaped in cross-section.

On an inner surface of the second flat plate portion 82, there are formed, in an outwardly projecting manner, a pair of thick ribs 98, 100 positioned on both sides thereof, a tab abutment 102, which abuts against the tab 30 of the catheter indwelling device 12 and is positioned substantially centrally in the widthwise direction of the second flat plate portion 82, a plate rib 104 positioned between the rib 98 and the tab abutment 102, a plate rib 106 positioned between the rib 100 and the tab abutment 102, and a pair of fitting projections 108, 110 (also referred to as second fitting projections 108, 110) that contact the second flat plate portion 44 of the inner needle hub 24.

The rib 98 is connected to the inner surface of the first side wall portion 84 and extends a predetermined length toward the proximal end side from the inner surface of the wall portion 78. The rib 100 is connected to the inner surface of the second side wall portion 86 and extends a predetermined length toward the proximal end side from the inner surface of the wall portion 78. In the present embodiment, the total length of each of the ribs 98, 100 is set to a length that is substantially one half the total length of the second flat plate portion 82.

The tab abutment 102 extends a predetermined length toward the proximal end side from the inner surface of the wall portion 78. The total length of the tab abutment 102 is set to be shorter than the total length of each of the first needle cover member abutments 88, 90, 92. This is because, in the puncture enabled state of the catheter indwelling device 12, the tab 30 is positioned more on the distal end side than the arm accommodating section 64. The tab abutment 102 projects to such a degree so as not to reach the opening edge of the proximal end side of the protective tube 70.

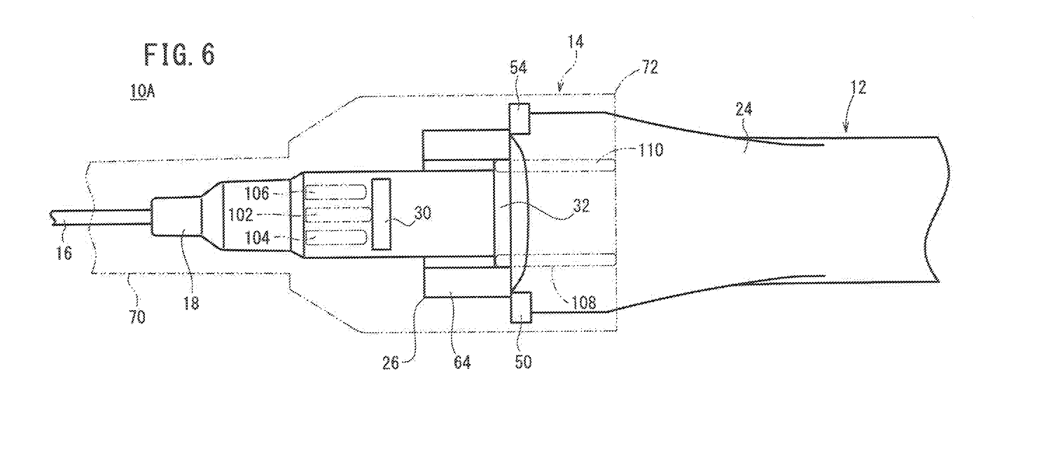

Each of the plate ribs 104, 106 extends a predetermined length toward the proximal end side from the inner surface of the wall portion 78. The total length of each of the plate ribs 104, 106 is set to be shorter than the total length of the tab abutment 102 (see FIG. 6). Each of the plate ribs 104, 106 projects to the opening edge of the proximal end side of the protective tube 70.

Each of the second fitting projections 108, 110 extends along the direction of extension of the protective tube 70 (the axial direction of the inner needle 22). The second fitting projection 108 faces the first fitting projection 94 of the first flat plate portion 80 in a state of extending from the distal end of the rib 98 to the proximal end of the second flat plate portion 82. The second fitting projection 110 faces the first fitting projection 96 of the first flat plate portion 80 in a state of extending from the distal end of the rib 100 to the proximal end of the second flat plate portion 82. Each of the second fitting projections 108, 110 has the same cross-sectional shape as the first fitting projections 94, 96.

On an inner surface of the first side wall portion 84, there are formed, in an outwardly projecting manner, a plurality of (two in the present embodiment) needle cover member abutments 112, 114 (also referred to as second needle cover member abutments 112, 114) that abut against the needle cover member 26 of the catheter indwelling device 12, a first guide portion (second engaging portion) 116 that is positioned substantially centrally in the widthwise direction (a direction along the vertical direction of FIG. 3) of the first side wall portion 84, a pair of first distal end side movement restricting portions 118, 120 that restrict movement of the first projection 50 of the catheter indwelling device 12 toward the distal end side with respect to the protector 14, and a pair of first proximal end side movement restricting portions 122, 124 that restrict movement of the first projection 50 toward the proximal end side.

Each of the second needle cover member abutments 112, 114 extends toward the proximal end side from the inner surface of the wall portion 78 by a length that is the same as the total length of the first needle cover member abutments 88, 90, 92 that are formed on the first flat plate portion 80. Each of the second needle cover member abutments 112, 114 projects to the opening edge of the proximal end side of the protective tube 70 toward the axis of the protective tube 70.

The first guide portion 116 extends to the proximal end of the first side wall portion 84 from the inner surface of the wall portion 78. The first guide portion 116 projects in a direction substantially perpendicular to the projecting direction of each of the first fitting projections 94, 96. The amount of projection thereof is set to a degree that is the same as the amount of projection of the first fitting projections 94, 96. Further, the width dimension of the first guide portion 116 is set to a dimension corresponding to the groove width of the first guide groove 52 that is formed in the first projection 50.

Each of the first distal end side movement restricting portions 118, 120 extends a predetermined length toward the proximal end side from the proximal end of each of the second needle cover member abutments 112, 114. The amount at which each of the first distal end side movement restricting portions 118, 120 project is set sufficiently shorter than the amount at which each of the second needle cover member abutments 112, 114 project.

The first proximal end side movement restricting portion 122 is positioned on the proximal end side of the first distal end side movement restricting portion 118, and extends toward the first flat plate portion 80 along the widthwise direction of the first side wall portion 84 from the first guide portion 116. In addition, the first proximal end side movement restricting portion 124 is positioned on the proximal end side of the first distal end side movement restricting portion 120, and extends toward the second flat plate portion 82 along the widthwise direction of the first side wall portion 84 from the first guide portion 116. The amount of projection of each of the first proximal end side movement restricting portions 122, 124 is sufficiently less than the amount of projection of each of the first distal end side movement restricting portions 118, 120, such that when the protector 14 is detached from the catheter indwelling device 12, the first projection 50 of the catheter indwelling device 12 can overcome the pair of first proximal end side movement restricting portions 122, 124.

On an inner surface of the second side wall portion 86, there are formed, in an outwardly projecting manner, a plurality of (two in the present embodiment) needle cover member abutments 126, 128 (also referred to as third needle cover member abutments 126, 128) that abut against the needle cover member 26 of the catheter indwelling device 12, a second guide portion (second engaging portion) 130 that is positioned substantially centrally in the widthwise direction of the second side wall portion 86, a pair of second distal end side movement restricting portions 132, 134 that restrict movement of the second projection 54 of the catheter indwelling device 12 toward the distal end side with respect to the protector 14, and a pair of second proximal end side movement restricting portions 136, 138 that restrict movement of the second projection 54 toward the proximal end side. As can be understood from FIG. 3, each of the third needle cover member abutments 126, 128 is constructed in the same manner as each of the second needle cover member abutments 112, 114.

The second guide portion 130 extends to the proximal end of the second side wall portion 86 from the inner surface of the wall portion 78. The amount at which the second guide portion 130 projects is set to the same amount as the amount at which the first guide portion 116 projects. Further, the second guide portion 130 projects in a direction substantially perpendicular to the projecting direction of each of the second fitting projections 108, 110. The width dimension of the second guide portion 130 is set to a dimension corresponding to the groove width of the second guide groove 56 that is formed in the second projection 54.

Each of the second distal end side movement restricting portions 132, 134 extends a predetermined length toward the proximal end side from the proximal end of each of the third needle cover member abutments 126, 128. The amount at which each of the second distal end side movement restricting portions 132, 134 projects is set to the same amount as the amount at which each of the first distal end side movement restricting portions 118, 120 projects.

The second proximal end side movement restricting portion 136 is positioned on the proximal end side of the second distal end side movement restricting portion 132, and extends toward the first flat plate portion 80 along the widthwise direction of the second side wall portion 86 from the second guide portion 130. In addition, the second proximal end side movement restricting portion 138 is positioned on the proximal end side of the second distal end side movement restricting portion 134, and extends toward the second flat plate portion 82 along the widthwise direction of the second side wall portion 86 from the second guide portion 130. The amount of projection of each of the second proximal end side movement restricting portions 136, 138 is sufficiently less than the amount of projection of each of the second distal end side movement restricting portions 132, 134, such that when the protector 14 is detached from the catheter indwelling device 12, the second projection 54 can overcome the pair of second proximal end side movement restricting portions 136, 138.

The catheter assembly 10A according to the present embodiment is constructed basically as described above. Next, operations and advantages of the catheter assembly 10A will be described.

During a stage in which the catheter assembly 10A is manufactured, the protector 14 is mounted on the distal end side of the catheter indwelling device 12 in the puncture enabled state, whereby the catheter assembly 10A is placed in an initial condition. At this time, when the distal end side of the catheter indwelling device 12 is inserted into the protector 14, the first guide portion 116 and the second guide portion 130 of the protector 14 enter respectively into the first guide groove 52 and the second guide groove 56 of the catheter indwelling device 12.

In addition, when the catheter indwelling device 12 is pushed further toward the distal end side of the protector 14, the first guide portion 116 slides along the wall surfaces that make up the first guide groove 52, and the second guide portion 130 slides along the wall surfaces that make up the second guide groove 56, whereupon the first projection 50 and the second projection 54 of the catheter indwelling device 12 respectively overcome the first proximal end side movement restricting portions 122, 124 and the second proximal end side movement restricting portions 136, 138 of the protector 14. The protector 14 then becomes mounted completely on the distal end side of the catheter indwelling device 12, thereby placing the catheter assembly 10A in the initial state.

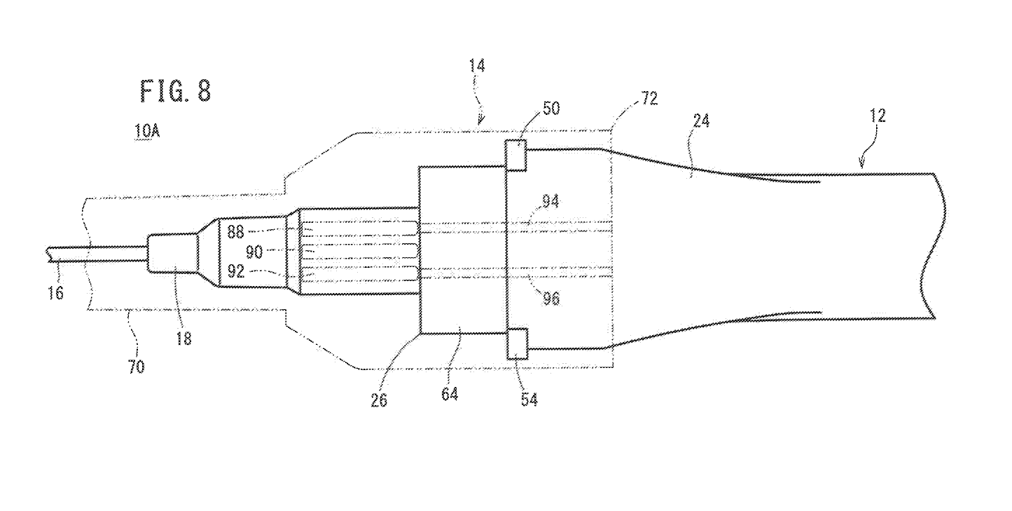

As shown in FIGS. 4 to 8, in the initial state of the catheter assembly 10A, the proximal end surface of the tab abutment 102 of the protector 14 abuts against the distal end surface of the tab 30 of the catheter indwelling device 12 (see FIGS. 4 and 6), and the proximal end surfaces of the needle cover member abutments 88, 90, 92, 112, 114, 126, 128 abut against the distal end surface of the arm accommodating section 64 of the needle cover member 26 (see FIGS. 4, 7 and 8). At this time, because the catheter hub 18 is fixed to the inner needle hub 24 through the needle cover member 26, movement thereof toward the proximal end side is restricted.

Further, the mounting portion 72 of the protector 14 is fitted over the distal end portion of the inner needle hub 24 of the catheter indwelling device 12 (see FIG. 5). More specifically, by abutment of the pair of first fitting projections 94, 96 against the outer surface of the first flat plate portion 42 of the inner needle hub 24, together with abutment of the pair of second fitting projections 108, 110 against the outer surface of the second flat plate portion 44 of the inner needle hub 24, the inner needle hub 24 is sandwiched between and is gripped by the first fitting projections 94, 96 and the second fitting projections 108, 110.

Furthermore, the proximal end surfaces of the first distal end side movement restricting portions 118, 120 and the second distal end side movement restricting portions 132, 134 of the protector 14 lie in close proximity to the respective distal end surfaces of the first projection 50 and the second projection 54 of the catheter indwelling device 12.

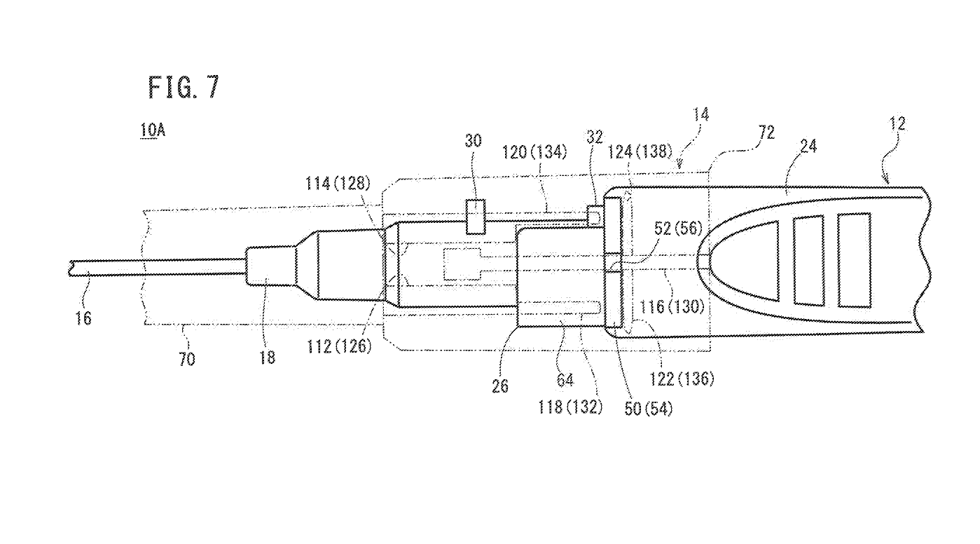

Together therewith, the distal end surfaces of the first proximal end side movement restricting portions 122, 124 and the second proximal end side movement restricting portions 136, 138 of the protector 14 abut against the respective proximal end surfaces of the first projection 50 and the second projection 54 of the catheter indwelling device 12 (see FIG. 7).

With the catheter assembly 10A, which is configured in the above-described manner, the tab abutment 102 and the needle cover member abutments 88, 90, 92, 112, 114, 126, 128 function as a movement prevention unit 140 (see FIG. 3) for preventing movement of the catheter 16 toward the distal end side with respect to the inner needle 22 in the initial state.

While it is necessary for the projecting length of the inner needle with respect to the catheter to be held constant in order to reliably insert the catheter into the patient's blood vessel, there is a concern that the length by which the inner needle projects with respect to the catheter may be changed due to relative movement in the axial direction between the inner needle and the catheter during transportation, sterilization, or the like of the catheter assembly.

However, according to the catheter assembly 10A of the present embodiment, the movement prevention unit 140 is provided for preventing movement of the catheter 16 toward the side of the tip 20 with respect to the inner needle 22, in a condition in which the distal end side of the inner needle 22 protrudes a predetermined length L from the catheter 16. Thus, in an initial state prior to use thereof, during transportation, sterilization, or the like of the catheter assembly 10A, relative movement between the catheter 16 and the inner needle 22 in the axial direction can be suppressed. Consequently, in the initial state, the length at which the inner needle 22 protrudes from the catheter 16 can be held constant (predetermined length L).

Further, in the initial state, since the proximal end surface of the tab abutment 102, which serves as the movement prevention unit 140, abuts against the distal end surface (the surface oriented toward the side where the tip 20 is positioned) of the tab 30 that is formed on the catheter hub 18, movement of the catheter 16 toward the tip 20 relative to the inner needle 22 can be prevented with a simple structure.

Moreover, since the proximal end surfaces of the needle cover member abutments 88, 90, 92, 112, 114, 126, 128, which serve as the movement prevention unit 140, abut against the distal end surface (the surface oriented toward the tip 20) of the arm accommodating section 64 that makes up the needle cover member 26, movement of the catheter 16 toward the tip 20 relative to the inner needle 22 can be reliably prevented with a simple structure.

According to the present embodiment, the pair of first fitting projections 94, 96 are formed to project on the inner surface of the first flat plate portion 80 of the mounting portion 72 that makes up the protector 14, together with the pair of second fitting projections 108, 110 being formed to project on the inner surface of the second flat plate portion 82 of the mounting portion 72. Additionally, each of the first fitting projections 94, 96 and the second fitting projections 108, 110 is formed to become narrower toward the direction in which they project. Therefore, the mounting portion 72 can be fitted externally over the inner needle hub 24 with a suitable fitting force. Consequently, in the initial state, since the protector 14 can be prevented from being displaced along the axial direction of the inner needle 22 with respect to the inner needle hub 24, the length at which the inner needle 22 protrudes from the catheter 16 can be more reliably held constant. Further, during use of the catheter indwelling device 12, the protector 14 can be detached from the inner needle hub 24 with an appropriate force.

Further, since the fitting projections 94, 96, 108, 110 respectively include outer surfaces that are arcuately shaped in cross-section, or stated otherwise, since they are formed to be substantially semicircular in cross-section, while the fitting force with respect to the inner needle hub 24 is appropriately suppressed, the rigidity of the fitting projections 94, 96, 108, 110 can suitably be increased. Thus, it can be made difficult for the fitting projections 94, 96, 108, 110 to become damaged.

Furthermore, since each of the fitting projections 94, 96, 108, 110 extends along the direction of extension of the protective tube 70 (the axial direction of the inner needle 22), at an initial stage in which the mounting portion 72 is to be fitted externally over the inner needle hub 24, the frictional force between the fitting projections 94, 96, 108, 110 and the inner needle hub 24 can be kept comparatively small. Consequently, it is easy for the mounting portion 72 of the protector 14 to be fitted externally over the inner needle hub 24, and an appropriate fitting force can be produced in a state in which the mounting portion 72 is fitted completely over the inner needle hub 24.

Further still, since the first fitting projection 94 and the second fitting projection 108 face each other mutually while sandwiching the inner needle hub 24 therebetween, and the first fitting projection 96 and the second fitting projection 110 face each other mutually while sandwiching the inner needle hub 24 therebetween, the inner needle hub 24 can be reliably gripped by the first fitting projections 94, 96 and the second fitting projections 108, 110. Thus, with a simple structure, an appropriate fitting force can be generated between the mounting portion 72 and the inner needle hub 24.

According to the present embodiment, the first guide groove 52 is formed in the first projection 50 of the inner needle hub 24, and the second guide groove 56 is formed in the second projection 54 of the inner needle hub 24. Additionally, the first guide portion 116 of a shape corresponding to the first guide groove 52 is formed to project on the inner surface of the first side wall portion 84 of the mounting portion 72, and the second guide portion 130 of a shape corresponding to the second guide groove 56 is formed to project on the inner surface of the second side wall portion 86 of the mounting portion 72. Therefore, the mounting portion 72 can be fitted externally over the inner needle hub 24 while the first guide portion 116 is made to slide along the wall surfaces that constitute the first guide groove 52, and the second guide portion 130 is made to slide along the wall surfaces that constitute the second guide groove 56. Consequently, the protector 14 can be easily mounted on the inner needle hub 24.

Further, in the initial state, since the distal end surfaces of the pair of first proximal end side movement restricting portions 122, 124 of the protector 14 are made to abut against the proximal end surface of the first projection 50 of the inner needle hub 24, and the distal end surfaces of the pair of second proximal end side movement restricting portions 136, 138 of the protector 14 are made to abut against the proximal end surface of the second projection 54 of the inner needle hub 24, a retracting movement of the catheter indwelling device 12 toward the proximal end side with respect to the protector 14 can be suppressed. Consequently, separation of the tab 30 that is formed on the catheter hub 18 away from the tab abutment 102, and separation of the arm accommodating section 64 of the needle cover member 26 away from the needle cover member abutments 88, 90, 92, 112, 114, 126, 128 can be suitably suppressed. Thus, a change in the projecting length of the inner needle 22 with respect to the catheter 16 can be suitably suppressed.

Furthermore, in the initial state, since the proximal end surfaces of the first distal end side movement restricting portions 118, 120 and the second distal end side movement restricting portions 132, 134 of the protector 14 lie in close proximity to the respective distal end surfaces of the first projection 50 and the second projection 54 of the catheter indwelling device 12, in the case that the catheter indwelling device 12 is pressed excessively toward the distal end side relative to the protector 14, the distal end surfaces of the first projection 50 and the second projection 54 come into abutment against the respective proximal end surfaces of the first distal end side movement restricting portions 118, 120 and the second distal end side movement restricting portions 132, 134. Thus, excessive entry of the catheter indwelling device 12 toward the distal end side relative to the protector 14 can be prevented. Therefore, damage to the tab 30 that is formed on the catheter hub 18 or the tab abutment 102 that is formed on the protector 14 can be prevented.

The catheter indwelling device 12 according to the present embodiment is equipped with the needle cover member 26 that is housed in the inner needle hub 24 in a condition capable of expanding toward the distal end side so as to cover the tip 20, and the catheter hub 18 is detachably disposed with respect to the distal end portion of the needle cover member 26. Therefore, when the inner needle 22 is withdrawn after the catheter 16 has been left indwelling in a blood vessel, the needle cover member 26 can be extended, whereby the tip 20 can be accommodated in the tip accommodating section 58. Owing thereto, inadvertent touching of the inner needle by the user can be prevented. Second Embodiment

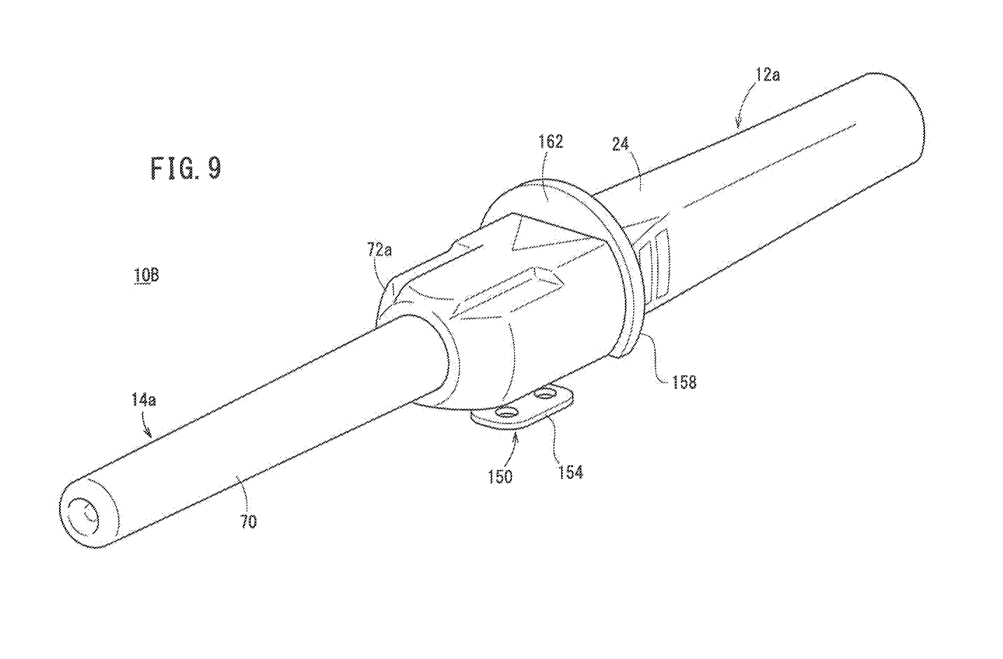

Next, a catheter assembly 10B according to a second embodiment of the present invention will be described with reference to FIGS. 9 to 15. In the catheter assembly 10B according to the present embodiment, constituent elements thereof, which are the same as those of the above-described catheter assembly 10A, are designated with the same reference characters, and detailed description of such features is omitted.

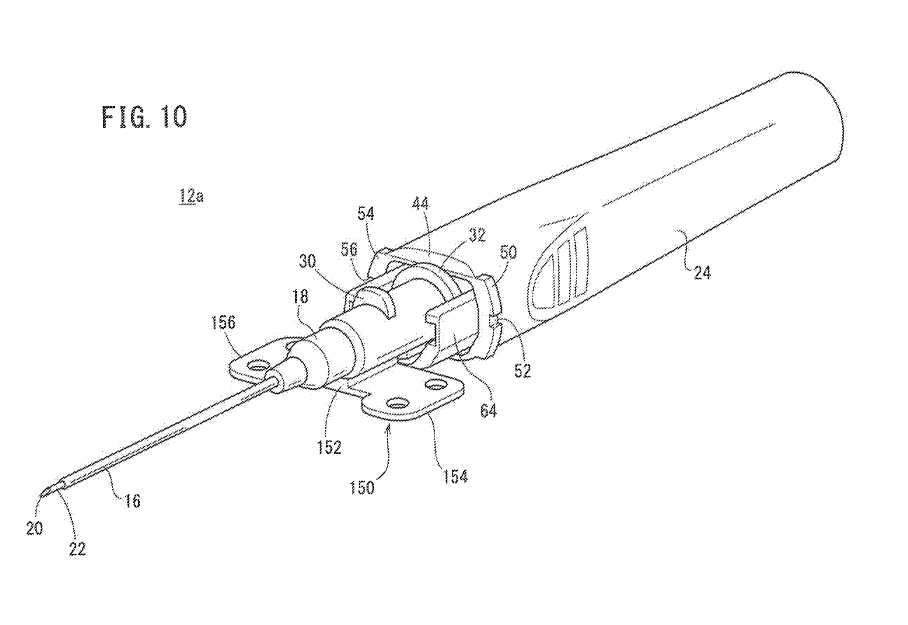

As shown in FIG. 9, the catheter assembly 10B according to the present embodiment is equipped with a catheter indwelling device 12a and a protector 14a. As shown in FIG. 10, the catheter indwelling device 12a includes a wing member 150 for facilitating adhering (fixing) of the catheter hub 18, which is exposed on the patient's skin in a state in which the catheter 16 has pierced the blood vessel, on the skin by tape or the like. The wing member 150 is disposed integrally on the catheter hub 18 on an opposite side of a side where the tab 30 is positioned, and extends outwardly on left and right sides thereof More specifically, the wing member 150 includes a support member 152 that projects outwardly from the catheter hub 18 on the opposite side of the side where the tab 30 is positioned and a pair of plate-shaped fixed wings 154, 156 that overhang from the support member 152 on left and right opposite sides.

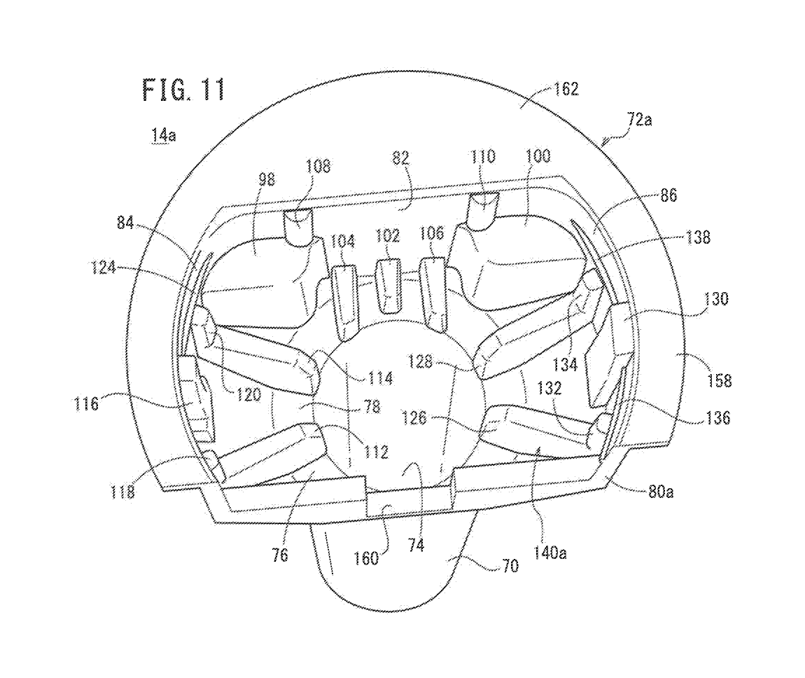

As shown in FIG. 11, the protector 14a includes the protective tube 70, a mounting portion 72a, and a flange 158 that projects out from and extends circumferentially on a proximal end of the mounting portion 72a. On a first flat plate portion 80a of the mounting portion 72a, a cutout 160 is formed for the purpose of avoiding the wing member 150 when the protector 14a is mounted on the distal end side of the catheter indwelling device 12a. More specifically, the needle cover member abutments 88, 90, 92 and the fitting projections 94, 96 of the first embodiment are not provided on the first flat plate portion 80a.

As can be understood from FIGS. 9 and 11, the amount at which the flange 158 projects from the second flat plate portion 82 is greater than the amount at which it projects from the first side wall portion 84 and the second side wall portion 86. The flange 158 is set to a size that enables a user to press and/or pull, with the fingers, a portion (widened portion 162) of the flange 158 that projects from the second flat plate portion 82.

Consequently, by pressing and/or pulling the widened portion 162 of the flange 158 with the fingers, the user can easily attach and detach the protector 14a with respect to the catheter indwelling device 12a. More specifically, if such a flange 158 were not provided, there would be a concern of the user's fingers interfering with the wing member 150 when the protector 14a was attached to or detached from the catheter indwelling device 12a. However, according to the present embodiment, since the flange 158 is disposed on the protector 14a, attachment and detachment of the protector 14a with respect to the catheter indwelling device 12a can be carried out smoothly.

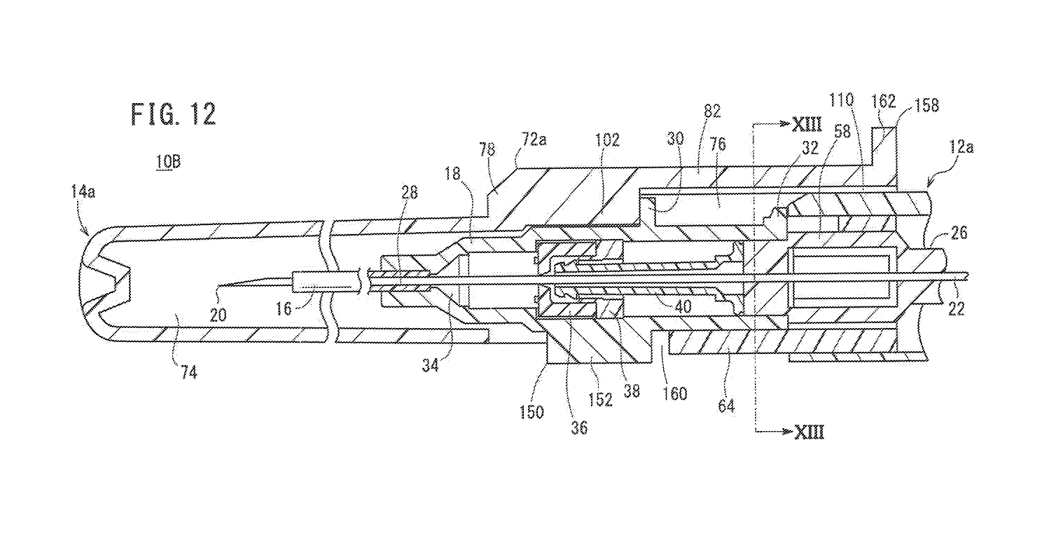

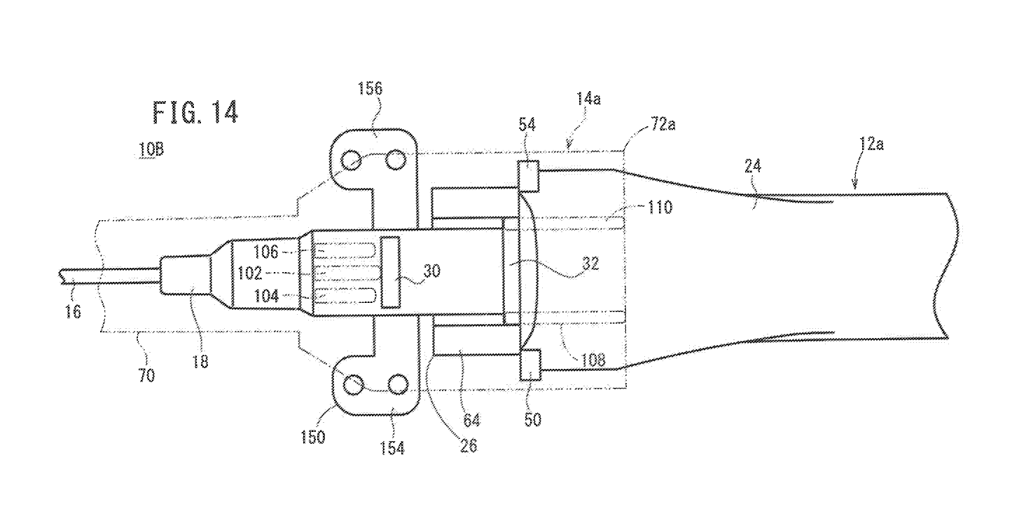

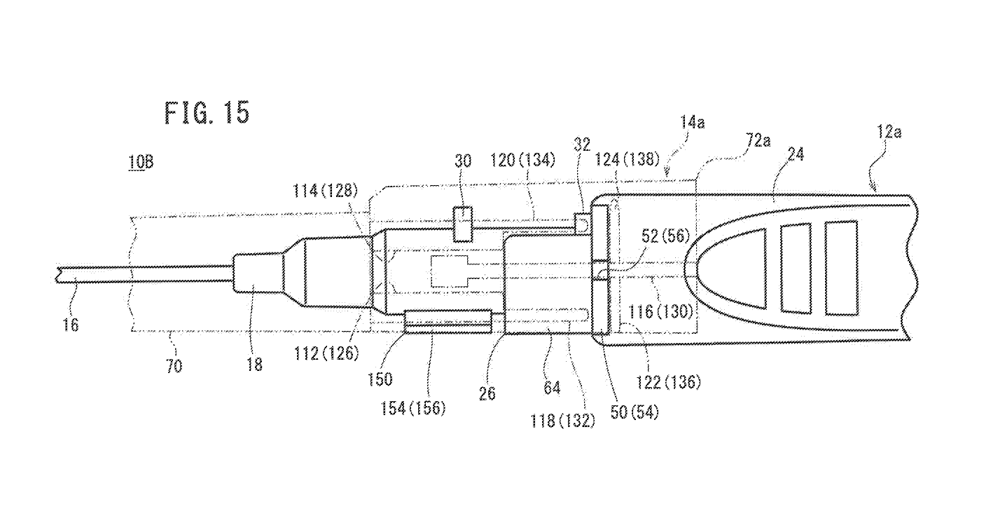

As shown in FIGS. 12 to 15, in the initial state of the catheter assembly 10B according to the present embodiment, the proximal end surface of the tab abutment 102 of the protector 14a abuts against the distal end surface of the tab 30 of the catheter indwelling device 12a (see FIGS. 12 and 14), and the proximal end surfaces of the needle cover member abutments 112, 114, 126, 128 abut against the distal end surface of the arm accommodating section 64 of the needle cover member 26 (see FIG. 15). At this time, because the catheter hub 18 is fixed to the inner needle hub 24 through the needle cover member 26, movement thereof toward the proximal end side is restricted.

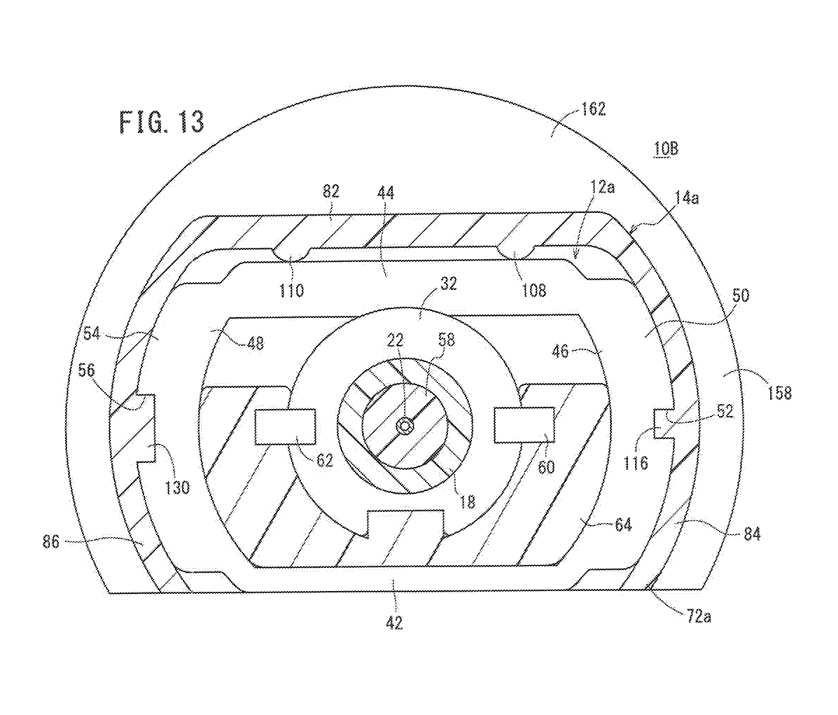

Further, the mounting portion 72a of the protector 14a is fitted over the distal end portion of the inner needle hub 24 of the catheter indwelling device 12a (see FIG. 13). More specifically, by abutment of the pair of fitting projections 108, 110 against the outer surface of the second flat plate portion 44 of the inner needle hub 24 and, moreover, by abutment of the first guide portion 116 against the wall surfaces that make up the first guide groove 52, and abutment of the second guide portion 130 against the wall surfaces that make up the second guide groove 56, the inner needle hub 24 is sandwiched between and is gripped by the fitting projections 108, 110, the first guide portion 116, and the second guide portion 130.

Furthermore, the proximal end surfaces of the first distal end side movement restricting portions 118, 120 and the second distal end side movement restricting portions 132, 134 of the protector 14a lie in close proximity to the respective distal end surfaces of the first projection 50 and the second projection 54 of the catheter indwelling device 12a. Together therewith, the distal end surfaces of the first proximal end side movement restricting portions 122, 124 and the second proximal end side movement restricting portions 136, 138 of the protector 14a abut against the respective proximal end surfaces of the first projection 50 and the second projection 54 of the catheter indwelling device 12a (see FIG. 15).

With the catheter assembly 10B, which is configured in the above-described manner, the tab abutment 102 and the needle cover member abutments 112, 114, 126, 128 function as the movement prevention unit 140a (see FIG. 11) for preventing movement of the catheter 16 toward the distal end side with respect to the inner needle 22 in the initial state.

With the catheter assembly 10B according to the present embodiment, although the wing member 150 is disposed on the catheter hub 18, because each of the first guide portion 116 and the second guide portion 130 projects substantially perpendicularly with respect to the direction in which the fitting projections 108, 110 project, the inner needle hub 24 can be sandwiched between and gripped by the fitting projections 108, 110, the first guide portion 116, and the second guide portion 130. Thus, with a simple structure, an appropriate fitting force can be generated between the mounting portion 72a and the inner needle hub 24. Further, the catheter assembly 10B according to the present embodiment can achieve the same advantages and effects of the above-described catheter assembly 10A according to the first embodiment.

Although preferred embodiments of the present invention have been described, the present invention is not limited to the above-described embodiments. It goes without saying that various modifications can be adopted therein without departing from the scope of the invention.

In the catheter assembly according to the present embodiment, each of the first engaging portions that are formed on the first projection and the second projection of the catheter indwelling device may be fabricated as protrusions, whereas each of the second engaging portions that are formed on the first side wall and the second side wall of the protector may be fabricated as recesses. Alternatively, both of the first engaging portions and the second engaging portions may be formed as protrusions. In this case, the mounting portion and the inner needle hub are moved along the axial direction of the inner needle in a state in which each of the first engaging portions of the catheter indwelling device and each of the second engaging portions of the protector are engaged, whereby the mounting portion can easily be fitted over the inner needle hub.

Furthermore, with the catheter assembly according to certain embodiments of the present invention, it is acceptable if at least one of the needle cover member abutments and the tab abutment is included to serve as the movement prevention unit.

What is claimed is:

1. A catheter assembly comprising:
an inner needle having a sharp tip at a distal end thereof;
a catheter in which the inner needle is disposed;
a catheter hub disposed on a proximal end side of the catheter and through which the inner needle is insertable;
an inner needle hub disposed on a proximal end side of the inner needle; and
a protector mounted detachably on the inner needle hub, the protector being configured to cover at least the tip of the inner needle;
wherein the protector comprises a movement prevention unit, the movement prevention unit being configured to prevent movement of the catheter toward the tip with respect to the inner needle in a state in which a distal end side of the inner needle protrudes a predetermined length from the catheter.

2. The catheter assembly according to claim 1, wherein a tab projects from an outer surface of the catheter hub, and the movement prevention unit includes a tab abutment configured to abut against a surface of the tab that is oriented toward the tip.

3. The catheter assembly according to claim 1, wherein:
the protector includes a hollow mounting portion configured to be detachably fitted externally over the inner needle hub; and
a fitting projection configured to contact the inner needle hub is formed in a projecting manner on an inner surface of the mounting portion.

4. The catheter assembly according to claim 3, wherein the fitting projection is formed to become narrower in the projecting direction of the fitting projection.

5. The catheter assembly according to claim 4, wherein the fitting projection includes an outer surface that is arcuately shaped in cross-section.

6. The catheter assembly according to claim 3, wherein the fitting projection extends along an axial direction of the inner needle.

7. The catheter assembly according to claim 3, wherein the fitting projection comprises a plurality of fitting projections, and the fitting projections face each other and sandwich the inner needle hub therebetween.

8. The catheter assembly according to claim 3, wherein:
the inner needle hub includes a first engaging portion, the first engaging portion being configured to guide the mounting portion along an axial direction of the inner needle; and
the mounting portion includes a second engaging portion formed on the inner surface thereof, the second engaging portion being configured to engage with the first engaging portion.

9. The catheter assembly according to claim 8, wherein:
either one of the first engaging portion and the second engaging portion is a protrusion that projects substantially perpendicularly to the projecting direction of the fitting projection; and
another one of the first engaging portion and the second engaging portion is a protrusion or a recess.

10. The catheter assembly according to claim 3, wherein:
the inner needle hub includes a projection, the projection projecting in a radially outward direction of the inner needle; and
the mounting portion includes a movement restricting portion formed on the inner surface thereof, the movement restricting portion being configured to engage with the projection and thereby restrict movement of the inner needle hub toward the proximal end side with respect to the protector in a state in which the mounting portion is fitted externally over the inner needle hub.

11. The catheter assembly according to claim 1, further comprising:
a needle cover member that is housed in the inner needle hub in an expandable condition toward the distal end side so as to cover the tip; and
wherein the catheter hub is disposed detachably with respect to the needle cover member.

12. The catheter assembly according to claim 11, wherein the movement prevention unit includes a needle cover member abutment configured to abut against a surface of the needle cover member that is oriented toward the tip.
